(19) United States
(12) Patent Application Publication
(10) Pub. No.: US 2026/0182923 A1
(43) Pub. Date: Jul. 2, 2026

(54) PHYSIOLOGICAL VALUE SENSING DEVICE AND SENSING METHOD THEREOF

(71) Applicant: Taiwan-Asia Semiconductor Corporation, Hsinchu City (TW)

(72) Inventors: Chia-Te Li, Hsinchu City (TW); Yu-Xin Huang, Hsinchu City (TW)

(21) Appl. No.: 19/357,394

(22) Filed: Oct. 14, 2025

(30) Foreign Application Priority Data
Dec. 31, 2024 (TW) 113151763

(51) Int. Cl. A61B 5/00; A61B 5/145; A61B 5/1455

(52) U.S. Cl. CPC A61B 5/7203; A61B 5/14532; A61B 5/1455; A61B 2560/0223

(57) ABSTRACT

The physiological value sensing device comprises a sensing module, a calibration module, and a signal processing module. The sensing module includes a sensing emission unit and a sensing receiving unit. The sensing emission unit emits a sensing light to a target tissue. The sensing light is reflected by the target tissue to the sensing receiving unit, generating a sensing signal. The calibration module includes a calibration emission unit and a calibration receiving unit. The calibration emission unit emits a calibration light to the calibration receiving unit to produce a reference signal. The signal processing module includes a denoising unit and a signal conversion unit. The denoising unit adjusts the sensing signal using the reference signal. The signal conversion unit extracts at least one peak value to form an extracted signal, selects a weight corresponding from a physiological value model, and narrows a bandwidth of the extracted signal to produce a physiological value signal.

### CROSS-REFERENCES TO RELATED APPLICATIONS

This application claims the benefit of priority to Taiwanese Patent Application No. 113151763 filed on Dec. 31, 2024, which is hereby incorporated by reference in its entirety.

### BACKGROUND OF THE INVENTION

### Field of the Invention

The present invention relates to a physiological value sensing device and a sensing method thereof, and more particularly to a non-invasive blood glucose sensing device and a sensing method thereof.

### Descriptions of the Related Art

Due to population aging and changes in modern lifestyles, the number of patients with diabetes worldwide is increasing rapidly. According to research by the World Health Organization (WHO), the number of global diabetes cases has quadrupled since 1990, and currently, more than 800 million adults are affected by diabetes.

Non-invasive blood glucose detection technology is a technique that measures blood glucose levels without the need to puncture the skin or extract blood samples. The most significant advantage of this technology is that it does not require the use of needles for puncture, making it more comfortable compared to traditional blood glucose testing methods and reducing patients'pain and discomfort. In addition, the non-invasive detection method also lowers the risk of infection for the subjects, thereby actively promoting public health. Moreover, such devices offer ease of operation, allowing users to perform measurements conveniently at home or in daily life without special training or assistance from medical professionals. Most importantly, non-invasive detection can provide continuous monitoring functionality, delivering more detailed data in real-time, which facilitates better management of related diseases for patients.

However, the accuracy of non-invasive detection technology is highly susceptible to environmental background influences. Particularly during optical signal detection, factors such as external temperature fluctuations, humidity changes, and ambient light interference may introduce additional noise. This noise can significantly degrade the quality of the reflected received optical signals, leading to measurement instability and deviations. Furthermore, unstable light intensity or excessive light scattering effects in the environment may further mask or distort the original detection signals, challenging the accuracy of non-invasive detection technology. To overcome the aforementioned issues, there is an urgent need in the industry for an innovative non-invasive physiological value sensing device and sensing method to improve these problems.

### SUMMARY OF THE INVENTION

The primary objective of the present invention is to provide an innovative physiological value sensing device and a sensing method thereof, which improves the issue of sensing accuracy being affected by background noise in known technologies.

To achieve the aforementioned objective, the present invention provides a physiological value sensing device, comprising a sensing module, a calibration module, and a signal processing module. The sensing module includes a sensing emission unit and a sensing receiving unit. The sensing emission unit emits a sensing light to a target tissue, and the sensing light is reflected by the target tissue to the sensing receiving unit to generate a sensing signal. The calibration module is disposed adjacent to the sensing module and includes a calibration emission unit and a calibration receiving unit. The calibration emission unit emits a calibration light to the calibration receiving unit to generate a reference signal. The signal processing module comprises a denoising unit and a signal conversion unit. The denoising unit adjusts the sensing signal into a denoised signal based on the reference signal. The signal conversion unit extracts at least one peak value from the denoised signal as an extracted signal, selects a weight corresponding to the at least one peak value from a physiological value model, and narrows a bandwidth of the extracted signal based on the weight to generate a physiological value signal.

In one embodiment of the physiological value sensing device of the present invention, the sensing emission unit and the calibration emission unit have the same structure, the sensing receiving unit and the calibration receiving unit have the same structure, and the sensing light and the calibration light have the same wavelength range.

In one embodiment of the physiological value sensing device of the present invention, the sensing emission unit and the calibration emission unit are light emitting diodes (LEDs), and the sensing receiving unit and the calibration receiving unit are photodiodes.

In one embodiment of the physiological value sensing device of the present invention, the wavelength range is from 1000 nanometers (nm) to 1800 nanometers (nm).

In one embodiment of the physiological value sensing device of the present invention, a center point of the sensing emission unit is spaced from a center point of the sensing receiving unit by a distance of 0.5 millimeters (mm) to 1.5 millimeters (mm).

In one embodiment of the physiological value sensing device of the present invention, the sensing module comprises a plurality of sensing emission units, and center points of the respective sensing emission units are spaced from each other by a distance of 0.5 millimeters (mm) to 1.5 millimeters (mm).

In one embodiment of the physiological value sensing device of the present invention, the signal conversion unit performs normalization or standardization on the denoised signal before extracting the at least one peak value to obtain the extracted signal.

In one embodiment of the physiological value sensing device of the present invention, the signal conversion unit performs z-score processing on the denoised signal.

In one embodiment of the physiological value sensing device of the present invention, the signal conversion unit performs normalization or standardization on the denoised signal and then shifts the denoised signal based on a reference value.

In one embodiment of the physiological value sensing device of the present invention, the at least one peak value is a relatively high value in the shifted denoised signal.

In one embodiment of the physiological value sensing device of the present invention, the at least one peak value is a highest value in the shifted denoised signal.

To achieve the aforementioned objectives, the present invention provides a physiological value sensing method, comprising: emitting a sensing light to a target tissue; receiving a reflected light reflected by the target tissue to generate a sensing signal; emitting a calibration light to a receiving unit adjacent to the target tissue to generate a reference signal; adjusting the sensing signal into a denoised signal based on the reference signal; extracting at least one peak value from the denoised signal as an extracted signal; and selecting a weight corresponding to the at least one peak value from a physiological value model, and narrowing a bandwidth of the extracted signal based on the weight to generate a physiological value signal.

In one embodiment of the physiological value sensing method of the present invention, the sensing light and the calibration light have the same wavelength range.

In one embodiment of the physiological value sensing method of the present invention, the wavelength range is from 1000 nanometers (nm) to 1800 nanometers (nm).

In one embodiment of the physiological value sensing method of the present invention, the method further comprises performing normalization or standardization on the denoised signal before the step of extracting at least one peak value from the denoised signal as an extracted signal.

In one embodiment of the physiological value sensing method of the present invention, the step of performing normalization or standardization on the denoised signal comprises performing z-score processing on the denoised signal.

In one embodiment of the physiological value sensing method of the present invention, the method further comprises shifting the denoised signal, after the step of performing normalization or standardization thereon, based on a reference value.

In one embodiment of the physiological value sensing method of the present invention, the at least one peak value is a relatively high value in the shifted denoised signal.

In one embodiment of the physiological value sensing method of the present invention, the at least one peak value is a highest value in the shifted denoised signal.

After referring to the drawings and the subsequent detailed description, those skilled in the art will readily understand other objectives of the present invention, as well as technical means and embodiments thereof.

### BRIEF DESCRIPTION OF THE DRAWINGS

FIG. 1 is a schematic diagram of a physiological value sensing device in accordance with an embodiment of the present invention;

FIG. 2 is a cross-sectional diagram of a sensing module and a calibration module in the physiological value sensing device in accordance with an embodiment of the present invention;

FIG. 3A is a top view schematic diagram of the sensing module in the physiological value sensing device in accordance with an embodiment of the present invention;

FIG. 3B is a top view schematic diagram of another aspect of the sensing module in the physiological value sensing device in accordance with an embodiment of the present invention; and

FIG. 4 is a schematic flowchart of a physiological value sensing method in accordance with an embodiment of the present invention.

### DETAILED DESCRIPTION OF THE PREFERRED EMBODIMENT

In the following description, the present invention will be explained with reference to various embodiments thereof. These embodiments of the present invention are not intended to limit the present invention to any specific environment, application, or particular method for implementations described in these embodiments. Therefore, the description of these embodiments is for illustrative purposes only and is not intended to limit the present invention. It shall be appreciated that, in the following embodiments and the attached drawings, a part of elements not directly related to the present invention may be omitted from the illustration, and dimensional proportions among individual elements and the numbers of each element in the accompanying drawings are provided only for ease of understanding but not to limit the present invention.

The physiological value sensing device of the present invention is a non-invasive continuous glucose monitoring (NICGM) device, configured to provide the subject with real-time, continuous, and long-term glucose concentration detection.

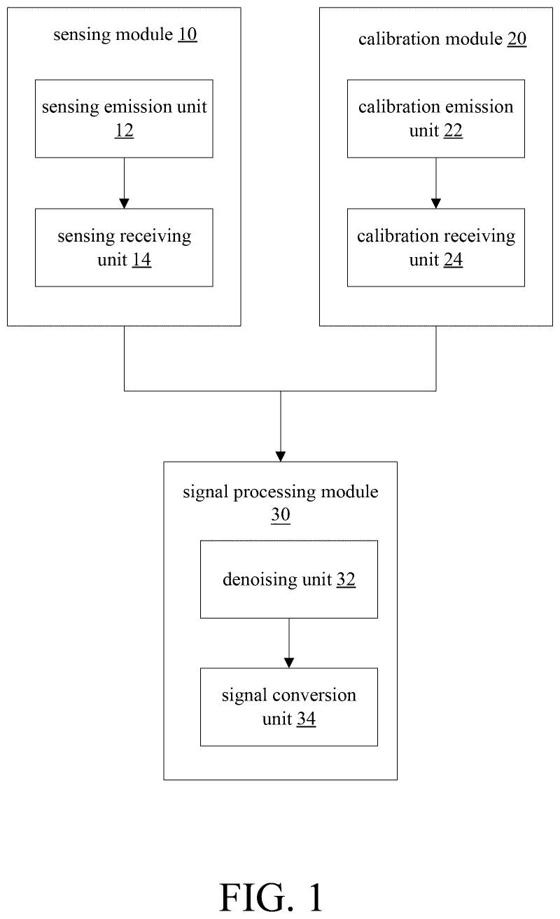

Referring to FIG. 1, which illustrates an embodiment of the present invention, a physiological value sensing device 1 is provided. As shown, the physiological value sensing device 1 comprises a sensing module 10, a calibration module 20, and a signal processing module 30.

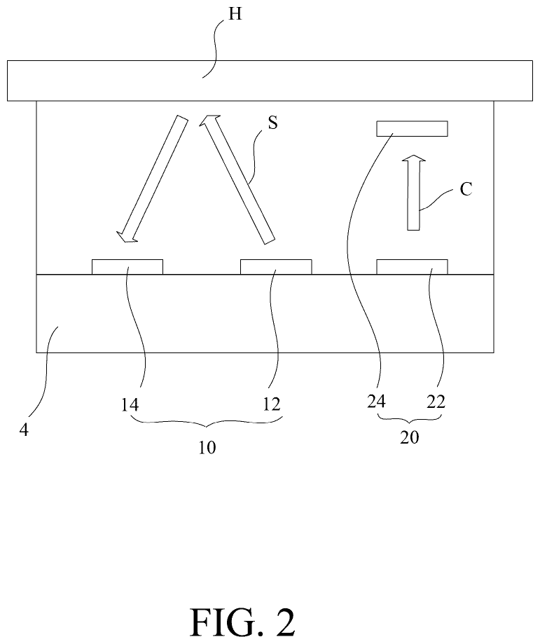

With reference also to the cross-sectional view of the sensing module 10 and the calibration module 20 in FIG. 2, the sensing module 10 and the calibration module 20 are electrically connected and disposed on a substrate 4. The sensing module 10 includes a sensing emission unit 12 and a sensing receiving unit 14. The sensing emission unit 12 emits a sensing light S to a target tissue H, such as human skin. The sensing light S undergoes reflection, absorption, and scattering through the target tissue H, thereby altering the optical path so that most of the sensing light S is diffusely reflected by the target tissue H to the sensing receiving unit 14, which converts the optical signal into an electrical sensing signal. The sensing signal carries physiological values, including the glucose level intended to be obtained by the present invention for subsequent processing and determination.

As shown in FIG. 2, the calibration module 20 of this embodiment is disposed adjacent to the sensing module 10. The calibration module 20 includes a calibration emission unit 22 and a calibration receiving unit 24. The calibration emission unit 22 emits a calibration light C to the calibration receiving unit 24 to generate a reference signal.

In this embodiment, the sensing emission unit 12 and the calibration emission unit 22 have the same structure and are both light emitting diodes (LEDs); the sensing receiving unit 14 and the calibration receiving unit 24 have the same structure and are both photodiodes. The sensing light and the calibration light have the same wavelength range, which is from 1000 nanometers (nm) to 1800 nanometers (nm).

During actual use of the physiological value sensing device 1 of the present invention, the sensing module 10 and the calibration module 20 operate simultaneously. Based on the structural configuration of the sensing module 10 and the calibration module 20 as described above, and due to the close proximity of the sensing module 10 and the calibration module 20, the light provided and received by both modules carries nearly identical noise caused by environmental or other factors, with the only difference being that the sensing signal also carries physiological values.

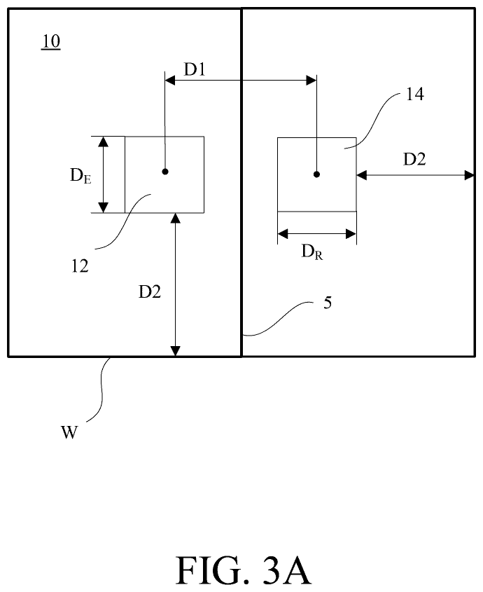

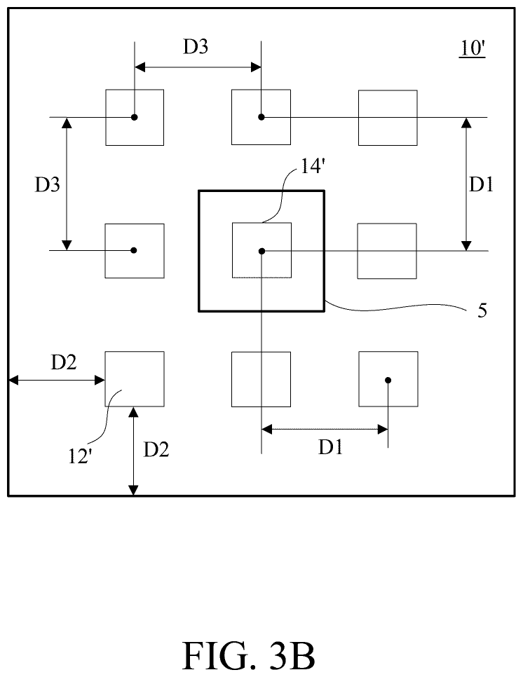

Please refer to FIG. 3A, which is a top view schematic diagram of the sensing module 10 of this embodiment. As shown in the figure, the distance D1 between the center point of the sensing emission unit 12 and the center point of the sensing receiving unit 14 is from 0.5 millimeters (mm) to 1.5 millimeters (mm). The distance D2 between either the sensing emission unit 12 or the sensing receiving unit 14 and a side wall W of the physiological value sensing device 1 is also from 0.5 mm to 1.5 mm. In addition, in this embodiment, the chip size D_{E} of the sensing emission unit 12 and the calibration emission unit 22 can be selected from 8 to 17 mils, and the chip size D_{R} of the sensing receiving unit 14 and the calibration receiving unit 24 can be selected from 12 to 27 mils. It should also be noted that the sensing module 10 of this embodiment further includes a microstructure 5, which is disposed between the sensing emission unit 12 and the sensing receiving unit 14 to prevent the light emitted by the sensing emission unit 12 from being directly received by the sensing receiving unit 14 without being diffusely reflected by the target tissue H.

In other aspects of this embodiment, please refer to FIG. 3B, which is a top view schematic diagram of the sensing module 10′ of another aspect of the physiological value sensing device 1′. As shown in the figure, the sensing module 10′ includes a plurality of sensing emission units 12′ and a sensing receiving unit 14′. Similarly, the distance D1 between the center point of any sensing emission unit 12′ and the center point of the sensing receiving unit 14′ is also from 0.5 mm to 1.5 mm. The distance D2 between any sensing emission unit 12′ and a side wall W of the physiological value sensing device 1 is also from 0.5 mm to 1.5 mm. The center points of each sensing emission unit 12′ are spaced apart from each other by a distance D3 of 0.5 mm to 1.5 mm. It should be noted that the arrangement and number of components in this embodiment are provided for illustrative purposes only and are not intended to limit the invention. Those skilled in the art can readily derive other variations. Furthermore, the aforementioned distances may differ in different directions and need not be the same. The sensing module 10′ of this embodiment also includes a microstructure 5, which will not be redundantly described here.

The signal processing module 30 includes a denoising unit 32 and a signal conversion unit 34. The denoising unit 32 adjusts the sensing signal into a denoised signal based on the reference signal, thereby correcting the physiological value measurement corresponding to the sensing signal, so that the denoised signal contains only information relevant to the desired physiological value to be measured, reducing the likelihood of misinterpretation.

After receiving the denoised signal, the signal conversion unit 34 first performs normalization or standardization. In this embodiment, the denoised signal undergoes z-score processing. Specifically, Z=(X−μ)/σ, where Z is the transformed data; X is the original data; μ is the mean of the dataset; and σ is the standard deviation of the dataset.

The signal conversion unit 34 performs a zero-point displacement on the z-score processed denoised signal based on a reference value. The signal conversion unit 34 then extracts at least one peak value from the denoised signal as an extracted signal, selects a weight corresponding to the at least one peak value from a physiological value model, and narrows the bandwidth of the extracted signal based on the weight to generate a physiological value signal. Preferably, the at least one peak value is a relatively high value in the shifted denoised signal, and more preferably, the at least one peak value is the highest value in the shifted denoised signal, thereby enabling subsequent signal conversion. The aforementioned weight used for narrowing the bandwidth of the extracted signal can be adjusted according to the bandwidth ratio of the peak value.

It should be noted that, in other embodiments of the present invention, the operations performed by the signal conversion unit—normalization or standardization processing, and shifting the denoised signal based on a reference value—may be omitted or executed in a different sequence.

When performing the aforementioned bandwidth narrowing operation, the signal conversion unit 34 needs to reference a physiological value model. This physiological value model is established through prior experimental procedures, in which an algorithm is used to adjust the signal to improve the accuracy of identifying blood glucose values corresponding to the signal. The physiological value sensing device of the present invention is applied to test samples, including a reference solution (such as milk) and a plurality of glucose solutions with different concentrations. The signals obtained from the physiological value sensing device respectively correspond to the reference solution and the various glucose solutions, and are organized to form the physiological value model, wherein the reference value corresponds to the reference solution.

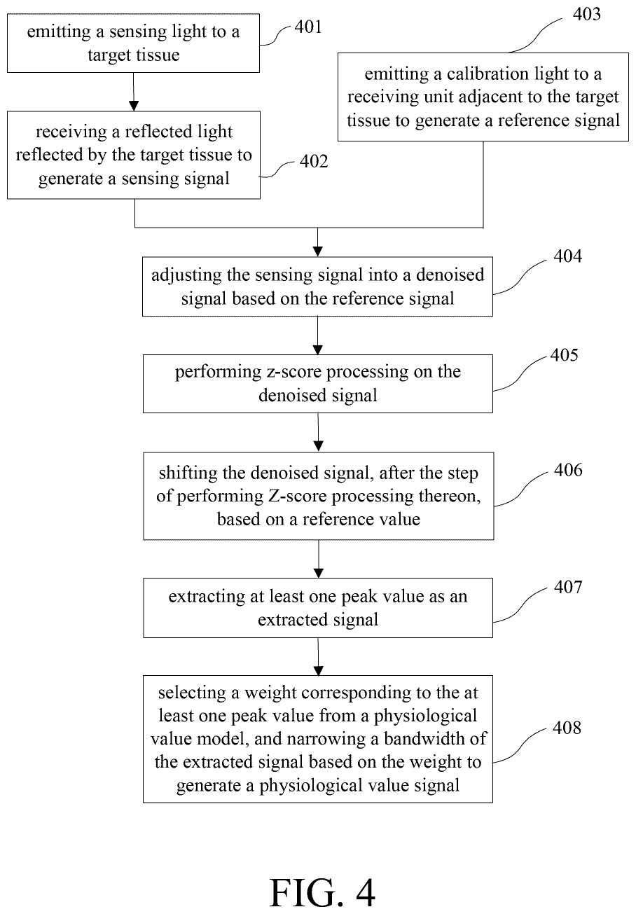

Based on the above content, please refer to the flowchart in FIG. 4. The physiological value sensing method disclosed in the present invention includes the following steps: In step 401, a sensing light is first provided to a target tissue. In step 402, a reflected light reflected by the target tissue is received to generate a sensing signal. In step 403, a calibration light is provided to a receiving unit adjacent to the target tissue to generate a reference signal. It should be noted that the sensing signal of steps 401 and 402 and the reference signal of step 403 are generated simultaneously or within a very short time interval. This allows the sensing signal and the reference signal to contain the same background noise. In addition, the sensing light and the calibration light have the same wavelength range, which is from 1000 nanometers (nm) to 1800 nanometers (nm).

Next, in step 404, the sensing signal is adjusted based on the reference signal to generate a denoised signal, that is, the portion of the sensing signal corresponding to the reference signal is removed to eliminate the influence of background noise and prevent subsequent value misjudgments. In step 405, the denoised signal is subjected to normalization or standardization processing. In this embodiment, the denoised signal is processed using z-score standardization, where Z=(X−μ)/σ, in which Z is the transformed data, X is the original data, μ is the mean of the dataset, and σ is the standard deviation of the dataset.

In step 406, the denoised signal that has been processed with the z-score is shifted based on a reference value. In this embodiment, the reference value corresponds to the value of the reference solution established in the aforementioned physiological value model. The signal corresponding to this reference solution is shifted to the origin, i.e., zero-point displacement is performed, thereby enhancing the accuracy of subsequent signal analysis and avoiding erroneous measurement results or misjudgments.

In step 407, at least one peak value of the denoised signal is extracted as an extracted signal. In step 408, a weight corresponding to at least one peak value is selected from the physiological value model, and based on the weight, the bandwidth of the extracted signal is narrowed to generate a physiological value signal. Preferably, the at least one peak value is a relatively high value in the shifted denoised signal, and more preferably, the at least one peak value is the highest value in the shifted denoised signal, to facilitate subsequent signal conversion. The resulting physiological value signal may then be used as a basis for determining blood glucose-related values of the target tissue.

In summary, the present invention provides a physiological value sensing device and a sensing method suitable for a non-invasive continuous glucose monitoring (NICGM) device. By disposing a calibration module adjacent to the sensing module, the invention can effectively acquire background environmental noise as a reference signal and perform real-time calibration to eliminate sensing errors. Moreover, by integrating a pre-established physiological value model, the sensing results are transformed into signals, significantly enhancing signal identification accuracy. The invention not only enables real-time, continuous, and long-term blood glucose concentration monitoring but also provides physiological values close to actual readings, thereby markedly improving the accuracy and reliability of non-invasive detection.

The above embodiments are used only to illustrate the implementations of the present invention and to explain the technical features of the present invention and are not used to limit the scope of the present invention. Any modifications or equivalent arrangements that can be easily accomplished by people skilled in the art are considered to fall within the scope of the present invention, and the scope of the present invention should be limited by the claims of the patent application.

What is claimed is:

1. A physiological value sensing device, comprising:
a sensing module, including a sensing emission unit and a sensing receiving unit, wherein the sensing emission unit emits a sensing light to a target tissue, and the sensing light is reflected by the target tissue to the sensing receiving unit to generate a sensing signal;
a calibration module, disposed adjacent to the sensing module, the calibration module including a calibration emission unit and a calibration receiving unit, wherein the calibration emission unit emits a calibration light to the calibration receiving unit to generate a reference signal; and
a signal processing module, including:
a denoising unit, configured to adjust the sensing signal into a denoised signal based on the reference signal; and
a signal conversion unit, configured to extract at least one peak value from the denoised signal as an extracted signal, select a weight corresponding to the at least one peak value from a physiological value model, and narrow a bandwidth of the extracted signal based on the weight to generate a physiological value signal.

2. The physiological value sensing device according to claim 1, wherein the sensing emission unit and the calibration emission unit have the same structure, the sensing receiving unit and the calibration receiving unit have the same structure, and the sensing light and the calibration light have the same wavelength range.

3. The physiological value sensing device according to claim 2, wherein the sensing emission unit and the calibration emission unit are light emitting diodes (LEDs), and the sensing receiving unit and the calibration receiving unit are photodiodes.

4. The physiological value sensing device according to claim 3, wherein the wavelength range is from 1000 nanometers (nm) to 1800 nanometers (nm).

5. The physiological value sensing device according to claim 4, wherein a center point of the sensing emission unit is spaced from a center point of the sensing receiving unit by a distance of 0.5 millimeters (mm) to 1.5 millimeters (mm).

6. The physiological value sensing device according to claim 4, wherein the sensing module comprises a plurality of sensing emission units, and center points of the respective sensing emission units are spaced from each other by a distance of 0.5 millimeters (mm) to 1.5 millimeters (mm).

7. The physiological value sensing device according to claim 1, wherein the signal conversion unit performs normalization or standardization on the denoised signal before extracting the at least one peak value to obtain the extracted signal.

8. The physiological value sensing device according to claim 7, wherein the signal conversion unit performs z-score processing on the denoised signal.

9. The physiological value sensing device according to claim 7, wherein the signal conversion unit performs normalization or standardization on the denoised signal and then shifts the denoised signal based on a reference value.

10. The physiological value sensing device according to claim 9, wherein the at least one peak value is a relatively high value in the shifted denoised signal.

11. The physiological value sensing device according to claim 9, wherein the at least one peak value is a highest value in the shifted denoised signal.

12. A physiological value sensing method, comprising:
emitting a sensing light to a target tissue;
receiving a reflected light reflected by the target tissue to generate a sensing signal;
emitting a calibration light to a receiving unit adjacent to the target tissue to generate a reference signal;
adjusting the sensing signal into a denoised signal based on the reference signal;
extracting at least one peak value from the denoised signal as an extracted signal; and
selecting a weight corresponding to the at least one peak value from a physiological value model, and narrowing a bandwidth of the extracted signal based on the weight to generate a physiological value signal.

13. The physiological value sensing method according to claim 12, wherein the sensing light and the calibration light have the same wavelength range.

14. The physiological value sensing method according to claim 13, wherein the wavelength range is from 1000 nanometers (nm) to 1800 nanometers (nm).

15. The physiological value sensing method according to claim 12, further comprising performing normalization or standardization on the denoised signal before the step of extracting at least one peak value from the denoised signal as an extracted signal.

16. The physiological value sensing method according to claim 15, wherein the step of performing normalization or standardization on the denoised signal comprises performing z-score processing on the denoised signal.

17. The physiological value sensing method according to claim 15, further comprising shifting the denoised signal, after the step of performing normalization or standardization thereon, based on a reference value.

18. The physiological value sensing method according to claim 16, wherein the at least one peak value is a relatively high value in the shifted denoised signal.

19. The physiological value sensing method according to claim 16, wherein the at least one peak value is a highest value in the shifted denoised signal.
